(19) United States
(12) Patent Application Publication
(10) Pub. No.: US 2004/0228883 A1
(43) Pub. Date: Nov. 18, 2004

(54) Prepackaged aqueous pharmaceutical formulation for the treatment of cardiac conditions containing at least two different active agents and method of formulation

(76) Inventor: Mitchell Karl, Boca Raton, FL (US)

Correspondence Address: RUDEN, MCCLOSKY, SMITH, SCHUSTER & RUSSELL, P.A., P.O. BOX 1900, FORT LAUDERDALE, FL, 33301, US

(21) Appl. No.: 10/785,169

(22) Filed: Feb. 23, 2004

Related U.S. Application Data
(60) Provisional application No. 60/449,470, filed on Feb. 21, 2003.

(51) Int. Cl. A61K 31/517; A61K 9/00

(52) U.S. Cl. 424/400

(57) ABSTRACT

Prepackaged aqueous pharmaceutical compositions for the treatment of cardiac conditions are disclosed including at least two different pharmacologically active agents for the treatment of a cardiac condition, a buffering agent to buffer the composition, and an osmotic-adjusting agent. Additionally, a method of forming a prepackaged aqueous pharmaceutical composition for the treatment of cardiac conditions is disclosed, the method including the steps of mixing at least two different pharmacologically active agents, adding a buffering agent to the mixed at least two different pharmacologically active agents, and adding an osmotic-adjusting agent to the mixed at least two different pharmacologically active agents.

### CROSS-REFERENCE TO RELATED APPLICATIONS

This application claims the benefit of U.S. Provisional Application Ser. No. 60/449,470, filed on Feb. 21, 2003, entitled PREPACKAGED AQUEOUS PHARMACEUTICAL FORMULATION FOR THE TREATMENT OF CARDIAC CONDITIONS CONTAINING AT LEAST TWO DIFFERENT ACTIVE AGENTS AND METHOD OF FORMULATION.

### TECHNICAL FIELD OF THE INVENTION

The present invention relates to compositions suitable for oral drug delivery. In particular, the present invention relates to prepackaged compositions of aqueous pharmaceutical compositions for the treatment of cardiac conditions comprising at least two different active agents, a buffering agent and an osmotic-adjusting agent, and further relates to a method for its use.

### BACKGROUND OF THE INVENTION

Congestive heart failure is a complex and heterogeneous disease state associated with decreased cardiac performance and increased pulmonary and peripheral oedema. Congestive heart failure results when the left, right or both ventricles fail to pump sufficient blood to meet the body's needs. An estimated 4 million people currently in the United States have congestive heart failure. While no single drug or drug class has proven to be ideal in treating this disease, vasodilator therapy constitutes a major approach in its clinical management.

Organic nitrate esters, such as nitroglycerin, isosorbide dinitrate, isosorbide-5-mononitrate, etc. are organic chemicals that contain the ONO₂ group. Nitrates are part of a family of vasodilators called nitrovasodilators and have enjoyed extensive use in cardiovascular therapy; but other members of this class, e.g., nitroprusside, molsidomine and organic nitrites are not organic nitrates. Nitrovasodilators such as isosorbide dinitrate and glyceryl trinitrate are useful in treating congestive heart failure because they cause a prompt reduction in preload and/or afterload, and relieve the venous congestion often associated with this disease.

Nitroglycerin, also referred to as trinitroglycerin or glycerin trinitrate, has also been used to treat angina pectoris for over 100 years. Nitroglycerin and other nitrovasodilators have been available for the treatment of angina pectoris and congestive heart failure in a number of different dosage forms for some time. These include sublingual, oral and buccal tablets as well as capsules, topical creams and ointments, patches, tapes, lingual sprays and intravenous solutions.

Transdermal nitroglycerin patches were introduced in recent years in an effort to overcome some of the disadvantages and inconveniences of other dosage forms. In particular, transdermal patches were formulated to provide increased systemic bioavailability as well as constant delivery of the drug over a 24 hour period or longer. Typically, the patches are applied once daily, either in the morning or evening, and changed daily at approximately the same time, and have become popular in the treatment of chronic, stable angina and congestive heart failure. However, the positive effects of these patches are often short lived. For example, it has been shown that nitroglycerin produces rapid hemodynamic tolerance (within several hours) in congestive heart failure after continuous administration either by intravenous or transdermal routes. Intermittent dosing with a regimen of 12 hours on/12 hours off can avoid development of tolerance but the effect of the previous dose is lost within 2 hours of drug withdrawal, leaving the patient unprotected during the majority of the “dose-off” period. Furthermore, a more frequent on/off dosing strategy (4 or 8 hour on/off cycles) was not successful in avoiding tolerance development. At present no dosage regimen with nitrovasodilators has been developed that can achieve the dual objectives of avoidance of hemodynamic tolerance while continuously maintaining their beneficial effects.

Conventional means for delivering biologically-active agents, including, but not limited to, pharmaceutical and therapeutic agents, to animals are often severely limited by chemical barriers and physical barriers imposed by the body. Oral delivery of many biologically-active agents would be the route of choice if not for chemical and physico-chemical barriers such as the extreme and varying pH in the gastro-intestinal (GI) tract, exposure to powerful digestive enzymes, and the impermeability of gastro-intestinal membranes to the active agent. Among the numerous agents that are not typically suitable for oral administration are biologically-active peptides such as calcitonin and insulin. Examples of other compounds that are affected by these physico-chemical barriers are polysaccharides and particularly mucopolysaccharides, including, but not limited to, heparin; heparinoids; antibiotics; and other organic substances. These agents are rapidly destroyed in the gastro-intestinal tract by acid hydrolysis, enzymes, or the like.

Prior methods for orally administering vulnerable pharmacological agents have relied on the co-administration of adjuvants (e.g., resorcinols and non-ionic surfactants such as polyoxyethylene oleyl ether and n-hexadecyl polyethylene ether) to increase artificially the permeability of the intestinal walls; and on the co-administration of enzymatic inhibitors (e.g., pancreatic trypsin inhibitor, diisopropylfluorophosphate (DFF) and trasylol) to inhibit enzymatic degradation. Liposomes have also been described as drug delivery systems for insulin and heparin. See, for instance, U.S. Pat. No. 4,239,754. However, broad spectrum use of the aforementioned drug delivery systems is precluded for reasons including: (1) the need to use toxic amounts of adjuvants or inhibitors; (2) the lack of suitable low MW cargoes; (3) the poor stability and inadequate shelf life of the systems; (4) the difficulties in manufacturing the systems; (5) the failure of the systems to protect the active ingredient; and (6) the failure of the systems to promote absorption of the active agent.

More recently, microspheres of artificial polymers or proteinoids of mixed amino acids have been described for delivery of pharmaceuticals. For example, U.S. Pat. No. 4,925,673 describes drug containing microsphere constructs as well as methods for their preparation and use. These proteinoid microspheres are useful for delivery of a number of active agents.

Cardiac trauma often occurs in medical patients. Many patients seek medical attention when experiencing chest pains, angina, myocardial infarction and other cardiac disorders. The standard treatment for such patients is to introduce liquid solutions intravenously into the patient's circulatory system. Such liquid solutions are available in a variety of compositions. Some liquid solutions contain vitamins and selected minerals and sugars. The liquid solutions also may contain specific pharmaceuticals as anti-coagulants, blood thinners, and the like. The patient may also receive dosages of inotropic agents such as dopamine, dobutamine or isuprel. The known inotropic agents increase the contractility of the heart and increase the heart rate, but also increase the oxygen demand of the heart. The inotropic agents may be included in the saline solutions that are available.

Esmolol hydrochloride is a short-acting beta-blocker used for treatment or prophylaxis of cardiac disorders in mammals. Most of the currently available beta-blockers are stable drugs that can be administered to cardiac patients over relatively long periods of time. However, it is often desirable in the critical care setting to quickly reduce heart work or improve rhythmicity during a cardiac crisis, e.g., during or shortly after a myocardial infarction. Conventional beta-blocking agents can be employed for such treatment, but their long durations of action can cause undesirable side effects.

Esmolol hydrochloride contains an ester functional group and possesses the typical beta-adrenergic blocking activity. However, it differs from conventional beta-blocking compound in that esmolol hydrochloride has a short duration in vivo due to the presence of the ester group. Thus, esmolol hydrochloride is advantageous compared to the conventional beta-blockers because of its unique short-acting activity. However, the ester group in esmolol hydrochloride is found to be unstable in an aqueous environment because of it extreme susceptibility to hydrolytic degradation.

The stability of esmolol in water is mediated by the rate of acid/base hydrolysis of the labile aliphatic methyl ester group. In the past, the rate of degradation of esmolol hydrochloride has been reduced by the use of acetate as a buffer, maintaining the pH as close to 5.0 as possible, minimizing the concentration of esmolol in the solution, and minimizing the concentration of buffer used. Prior art formulations maintain a reasonably long shelf-life, however, they are packaged in glass vials or ampules, and suffer from severe degradation upon autoclaving. As a result, prior art formulations are prepared aseptically. C.f. U.S. Pat. No. 4,857,552. However, regulatory authorities typically prefer terminal sterilization as a way of reducing microbiological burden and to ensure the safety of the finished product.

In addition, the formulation disclosed in U.S. Pat. No. 4,857,552 is a small volume injectable formulation. For the purposes of intravenous infusion, the disclosed formulation must be further diluted in pharmaceutically acceptable diluents prior to use. This creates a potential opportunity for calculation or dilution error in a hospital setting. Additionally, microbiological contamination of the product during dilution/aseptic handling is of primary concern.

Thus, there is still a need in the art for simple and inexpensive oral delivery systems for drug compositions that are easily prepared and that can deliver a broad range of biologically-active agents.

### SUMMARY OF THE INVENTION

The present invention eliminates the above-mentioned needs for a simple and inexpensive oral delivery system for drug compositions by providing prepackaged aqueous pharmaceutical compositions for the treatment of cardiac conditions.

In accordance with the present invention, there are provided prepackaged aqueous pharmaceutical compositions for the treatment of cardiac conditions comprising at least two different pharmacologically active agents for the treatment of a cardiac condition, a buffering agent to buffer the composition, and an osmotic-adjusting agent.

The present invention is additionally directed to a method of forming a prepackaged aqueous pharmaceutical composition for the treatment of cardiac conditions, the method comprising the steps of mixing at least two different pharmacologically active agents, adding a buffering agent to the mixed at least two different pharmacologically active agents, and adding an osmotic-adjusting agent to the mixed at least two different pharmacologically active agents.

The present invention is further directed to a process for the administration of a prepackaged aqueous solution or dispersion of at least two different pharmacologically active agents for the treatment of a cardiac condition, comprising selecting a predetermined dosage amount for each of the at least two different pharmacologically active agents, the dosage amounts selected based upon patient characteristics, mixing the dosage amounts with a buffering agent and an osmotic-adjusting agent, the pharmacologically active agents having stability with one another in aqueous solution, and providing the pharmacologically active agents having stability with one another in aqueous solution to a suitable patient for oral administration.

### DETAILED DESCRIPTION OF THE PREFERRED EMBODIMENT

Common cardio ailments often require fairly standard combination of medications and dosages. Dose variability may depend on certain patient factors, but yield predictable finite and routine adjustments. Physicians acceptance of combination pill therapy has been limited by heretofor very restricted and limited dosing combinations that do not allow for patient factors that might necessitate dose adjustments. The inadequate available variations are viewed as limiting their utility in clinical practice.

However, if this obstacle could be circumvented and if the physician had multiple options to customize combination pre-packaged therapy for the individual patient based on common but clinically important differences, then physicians would likely embrace the concept.

Patients frequently complain about the cost of each of their medications and look for substitutes for particularly costly ones in their regimen. Patents often run out of, forget to take some of, have difficulty swallowing medications. Patients may question the validity of a multiple drug regime and/or feel overmedicated by the number of pills they must swallow.

These problems are addressed by a prepackaged combination of daily cardio medication in liquid form tailored to specific ranges for use by individual patients having differing characteristics and selected from a chart by the patient's physician.

For example, combo “A” might contain the most commonly prescribed dosage of a standard regime for congestive heart failure:

| | |
|---|---|
| Digoxin/Lanoxin | 25 mg |
| Lasix/Furosamide | 20 mg |
| Potassium Chloride | 10 mg |
| Altace/Rampiril | 5 mg |

For example, those with conduction system disease, bradycardia or renal disease, Digoxin is omitted from the regimen in combo “A”-2. For those with low blood pressure the Altace dosage is reduced or eliminated. Greater diuretic dosages would be provided in the combination for those who have such a need.

Although not every patient's needs fit neatly and exactly into an offered combination, a combination that very closely fits the patient's needs could be selected as most suitable and could then modified by the addition of one or two pills to the regime. This would not therefore diminish the utilization of this concept as it could be applied in whole or in part to most patients.

In all cases, the physician would be able to select the desired combination and dosing for the patient; and the patient would enjoy the greater simplicity, decreased cost, easier compliance, and greater confidence associated with prepackaged daily dosing.

Packaging could take the form of a plastic disposable dispenser with twist-off cap. Medication could be in the form of a combined liquid/elixir. Nutraceuticals and vitamins may be included in drug regime if sufficient evidence supports their use.

### EXAMPLES

Cardio Combo A - for congestive heart failure

| | |
|---|---|
| A1 | Digoxin-.25 mg; Lasix-20 mg; KLL-10 meg; Altace-5 mg |
| A2 | Digoxin-.125 mg; Lasix-40 mg; KLL-10 meg; Altace-2.5 mg |
| A3 | Lasix-40 mg; KLL-20 mg; Altace-2.5; CQ10 |
| A4 | Digoxin-.125 mg; Lasix-20 mg; KLL-10 mg; CQ10 |
| A5 | Digoxin-.125 mg; Lasix-40 mg; CQ10 |
| A6 | Digoxin-.25 mg; Lasix-20 mg; KLL-10 mg; Altace-2.5 + Coreg-3.1 |
| | (a single 3.1 mg core pill to be taken later in the |
| | day). |
| A7 | Same as A6 with 6.25 mg coreg |
| A8 | Same as A6 with 12.5 mg coreg |
| A9 | Same as A6 with 25 mg coreg |

Cardio Combo B- for coronary artery disease:

| | |
|---|---|
| B1 | ASA-81 mg; Statin; B complex, Vitamin E |
| B2 | Same as B1, but with greater Statin dose |
| B3 | Same as B2 with addition of niacin |
| B4 | Same as B1 plus nitrate |
| B5 | Same as B4 plus beta blocker |
| B6 | Same as B5 plus calcium channel blocker |

Although only a few exemplary embodiments of the present invention have been described in detail above, those skilled in the art will readily appreciate that numerous modifications are to the exemplary embodiments are possible without materially departing from the novel teachings and advantages of this invention. Accordingly, all such modifications are intended to be included within the scope of this invention as defined in the following claims.

What is claimed is:

1. A prepackaged aqueous pharmaceutical composition for the treatment of cardiac conditions, comprising:
at least two different pharmacologically active agents for the treatment of a cardiac condition;
a buffering agent to buffer said composition; and
an osmotic-adjusting agent.

2. A method of forming a prepackaged aqueous pharmaceutical composition for the treatment of cardiac conditions, said method comprising the steps of:
mixing at least two different pharmacologically active agents;
adding a buffering agent to said mixed at least two different pharmacologically active agents; and
adding an osmotic-adjusting agent to said mixed at least two different pharmacologically active agents.

3. A process for the administration of a prepackaged aqueous solution or dispersion of at least two different pharmacologically active agents for the treatment of a cardiac condition, comprising:
selecting a predetermined dosage amount for each of said at least two different pharmacologically active agents, said dosage amounts selected based upon patient characteristics;
mixing said dosage amounts with a buffering agent and an osmotic-adjusting agent, said pharmacologically active agents having stability with one another in aqueous solution; and
providing said pharmacologically active agents having stability with one another in aqueous solution to a suitable patient for oral administration.

4. The composition according to claim 1 wherein said at least two different pharmacologically active agents are selected from the group consisting of diuretics, cardiac glycocides, beta blockers, nitrates, antiplatelets, vitamins, nitroceuticals, angiotensin converting enzyme inhibitors, and calcium channel clockers.

5. The composition according to claim 1 wherein said buffering agent comprises at least one of acetate, glutamate, citrate, tartrate, benzoate, lactate, gluconate, phosphate and glycine.

6. The composition according to claim 1 wherein said osmotic-adjusting agent comprises at least one of sodium chloride, dextrose, sodium bicarbonate, calcium chloride, potassium chloride, sodium lactate, Ringer's solution and lactated Ringer's solution.

7. The composition according to claim 2 wherein said at least two different pharmacologically active agents are selected from the group consisting of diuretics, cardiac glycocides, beta blockers, nitrates, antiplatelets, vitamins, nitroceuticals, angiotensin converting enzyme inhibitors, and calcium channel blockers.

8. The composition according to claim 2 wherein said buffering agent comprises at least one of acetate, glutamate, citrate, tartrate, benzoate, lactate, gluconate, phosphate and glycine.

9. The composition according to claim 2 wherein said osmotic-adjusting agent comprises at least one of sodium chloride, dextrose, sodium bicarbonate, calcium chloride, potassium chloride, sodium lactate, Ringer's solution and lactated Ringer's solution.

10. The composition according to claim 3 wherein said at least two different pharmacologically active agents are selected from the group consisting of diuretics, cardiac glycocides, beta blockers, nitrates, antiplatelets, vitamins, nitroceuticals, angiotensin converting enzyme inhibitors, and calcium channel clockers.

11. The composition according to claim 3 wherein said buffering agent comprises at least one of acetate, glutamate, citrate, tartrate, benzoate, lactate, gluconate, phosphate and glycine.

12. The composition according to claim 3 wherein said osmotic-adjusting agent comprises at least one of sodium chloride, dextrose, sodium bicarbonate, calcium chloride, potassium chloride, sodium lactate, Ringer's solution and lactated Ringer's solution.
